(19) United States
(12) Patent Application Publication
(10) Pub. No.: US 2026/0224120 A1
(43) Pub. Date: Aug. 6, 2026

(54) Finger Cuff Assembly Having Barometric Pressure Sensors and Optical Sensors for Measuring Blood Pressure

(71) Applicant: Google LLC, Mountain View, CA (US)

(72) Inventors: Ravi Narasimhan, Los Altos, CA (US); Samuel Wei Sheng, Saratoga, CA (US); Paul Joseph Silberschatz, San Francisco, CA (US)

(21) Appl. No.: 19/149,711

(22) PCT Filed: Jan. 25, 2023
(86) PCT No.: PCT/US2023/011499

§ 371 Date: Jul. 21, 2025

(51) Int. Cl. A61B 5/022; A61B 5/021; A61B 5/1455

(52) U.S. Cl. CPC A61B 5/02241; A61B 5/02116; A61B 5/14551; A61B 2562/046; A61B 2562/16; A61B 2562/222

(57) ABSTRACT

A finger cuff assembly includes a finger cuff having a cavity defining an inner surface and an array of barometric pressure sensors arranged on the inner surface. The array of barometric pressure sensors is configured to map a pressure surface near an artery in the finger to provide a plurality of data channels for measuring the blood pressure. The finger cuff assembly also includes one or more optical sensors arranged on the inner surface adjacent to the array of barometric pressure sensors and an inflatable bladder arranged with the array of barometric pressure sensors, wherein inflating the bladder increases a pressure on the array of barometric pressure sensors against the finger. The finger cuff assembly also includes a control module having circuitry for receiving data from the plurality of data channels and estimating the blood pressure using the data. As the pressure on the array of barometric pressure sensors increases, pulses from the artery are detected on the plurality of data channels and used by the control module for estimating the blood pressure.

### FIELD

The present disclosure relates generally to systems and methods of measuring blood pressure, and more particularly, to a finger cuff assembly having an array of barometric pressure sensors and one or more optical sensors for measuring blood pressure.

### BACKGROUND

Non-invasive blood pressure (BP) measurements are typically performed using external cuffs that apply pressure to one or more arteries of a patient, with the response of the arteries being observed to determine the BP. Auscultatory and oscillometric BP cuffs use this technique to obtain discrete (non-continuous) BP measurements. The volume clamp method with a finger cuff uses related techniques to obtain continuous BP measurements.

Cuffless BP measurement techniques aim to reduce patient burden and increase BP monitoring frequency. However, most cuffless BP technologies rely on optical signals (e.g., photoplethysmography (PPG)) at a peripheral body site (e.g., wrist or finger) and often another physiological signal such as electrocardiogramar ballistocardiography (BCG). The time difference of the pulse signal between the ECG or BCG to the PPG signal is referred to as a pulse arrival time (PAT) or a pulse transit time (PTT), respectively. Cuffless BP methods use the fact that PAT and PTT are often correlated with BP. Using an arm cuff calibration, these cuffless approaches attempt to track changes in the patient's BP over time. However, the relatively low correlation of PAT and PTT with BP and the presence of other interfering factors make cuffless BP approaches unsuitable for clinically accurate BP monitoring. As such, techniques that use the shape of the PPG waveform (known as pulse wave analysis) to estimate BP have been used but suffer from similar shortcomings as other cuffless BP methods based on PAT or PTT.

Accordingly, the present disclosure is directed to improved systems and methods of measuring blood pressure. In particular, the present disclosure is directed to a finger cuff assembly having an array of barometric pressure and optical sensors and one or more optical sensors for measuring blood pressure of a patient.

### SUMMARY

Aspects and advantages of embodiments of the present disclosure will be set forth in part in the following description, or can be learned from the description, or can be learned through practice of the embodiments.

In an aspect, the present disclosure is directed to a finger cuff assembly for measuring blood pressure of a patient. The finger cuff assembly includes a finger cuff having a cavity defining an inner surface configured to engage a finger of the patient. The finger cuff assembly also includes an array of barometric pressure sensors arranged on the inner surface of the cavity of the finger cuff. The array of barometric pressure sensors is configured to map a pressure surface near an artery in the finger to provide a plurality of data channels for measuring the blood pressure. The finger cuff assembly also includes one or more optical sensors arranged on the inner surface of the cavity of the finger cuff adjacent to the array of barometric pressure sensors and an inflatable bladder arranged with the array of barometric pressure sensors, wherein inflating the inflatable bladder increases a pressure on the array of barometric pressure sensors against the finger of the patient. Further, the finger cuff assembly includes a control module having circuitry for receiving data from the plurality of data channels and estimating the blood pressure using the data from the plurality of data channels. Thus, as the pressure on the array of barometric pressure sensors increases, pulses from the artery are detected on the plurality of data channels and used by the control module for estimating the blood pressure.

In an embodiment, the array of barometric pressure sensors is encapsulated in an elastomer material to provide a force transfer medium between the finger and the array of barometric pressure sensors.

In an embodiment, the elastomer material may include a silicone gel, silicone oil, or mineral oil. In another embodiment, the optical sensor(s) may include at least one of one or more pulse oximetry sensors, one or more thermal sensors, or one or more photoplethysmogram (PPG) sensors.

In another embodiment, the finger cuff assembly includes a micropump for transferring ambient air to the inflatable bladder, wherein the circuitry of the control module is further configured to control the micropump.

In another embodiment, the finger cuff assembly includes a housing for housing the control module and the micropump. In further embodiments, the housing is secured to the finger cuff and rests against at least one the finger or a palm of the patient when the finger cuff is positioned on the finger of the patient. In additional embodiments, the housing is separate from the finger cuff. In an embodiment, the housing is securable to a wrist of the patient.

In an embodiment, the finger cuff assembly includes one or more cables electrically coupling the circuitry of the control module to the array of barometric pressure sensors.

In another embodiment, the circuitry of the control module is further configured to: store the data from the plurality of data channels; and estimate the blood pressure using the pulses from the artery detected on the plurality of data channels and stored in the circuitry of the control module. In an embodiment, the pulses from the artery detected on the plurality of data channels include one or more distinct features that vary throughout a pressure sweep. In such embodiments, the distinct feature(s) include at least one of pulse amplitude or pulse morphology.

In another aspect, the present disclosure is directed to a method for estimating blood pressure of a patient via a finger cuff assembly. The method includes positioning a finger cuff of the finger cuff assembly on a finger of the patient such that an array of barometric pressure sensors and one or more optical sensors arranged on an inner surface of the finger cuff contact the finger of the patient. The method also includes inflating an inflatable bladder of the finger cuff assembly to increase a pressure on the array of barometric pressure sensors against the finger of the patient. Further, the method includes mapping, via the array of barometric pressure sensors, a pressure surface near an artery in the finger to provide a plurality of data channels relating to the blood pressure. Moreover, the method includes receiving, via a control module of the finger cuff assembly, data from the plurality of data channels and data from the one or more optical sensors. In addition, the method includes estimating the blood pressure of the patient as a function of the pressure on the array of barometric pressure sensors and pulses from the artery detected on the plurality of data channels and the data from the one or more optical sensors.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1 illustrates a perspective view of an embodiment of a finger cuff assembly according to the present disclosure;

FIG. 2 illustrates another perspective view of the finger cuff assembly of FIG. 1;

FIG. 3A illustrates a cross-sectional view of the finger cuff assembly of FIG. 1;

FIG. 3B illustrates a front view of an array of sensors of the finger cuff assembly of FIG. 3A;

FIG. 3C illustrates a perspective view of an array of sensors on an inner surface of the finger cuff assembly of FIG. 3A;

FIG. 4 illustrates a top view of the finger cuff assembly of FIG. 1;

FIG. 5 illustrates a bottom view of the finger cuff assembly of FIG. 1;

FIG. 6 illustrates a schematic diagram of an embodiment of components housed in a housing of a finger cuff assembly according to the present disclosure;

FIG. 7 illustrates a graph of an embodiment of pressure (y-axis) versus time (x-axis) to illustrate a response of a sensor array of a finger cuff assembly according to the present disclosure;

FIG. 8 illustrates a graph of an embodiment of pressure (y-axis) versus time (x-axis) to illustrate a pulsatile response of a sensor array of a finger cuff assembly according to the present disclosure;

FIG. 9 illustrates a graph of an embodiment of a response of an optical sensor during a pressure sweep of a finger cuff assembly according to the present disclosure; and

FIG. 10 illustrates a flow diagram of a process of measuring blood pressure according to the present disclosure.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

### Overview

As mentioned, cuffless BP measurement techniques aim to reduce patient burden and increase BP monitoring frequency. However, most cuffless BP technologies rely on optical signals (photoplethysmography (PPG)) at a peripheral body site (e.g., wrist or finger) and often another physiological signal such as electrocardiogramar ballistocardiography (BCG). The time difference of the pulse signal between the ECG or BCG to the PPG signal is referred to as a pulse arrival time (PAT) or a pulse transit time (PTT), respectively. Cuffless BP methods use the fact that PAT and PTT are often correlated with BP. Using an arm cuff calibration, these cuffless approaches attempt to track changes in the patient's BP over time. However, the relatively low correlation of PAT and PTT with BP and the presence of other interfering factors make cuffless BP approaches unsuitable for clinically accurate BP monitoring. As such, techniques that use the shape of the PPG waveform (known as pulse wave analysis) itself to estimate BP have been used but suffer from similar shortcomings as other cuffless BP methods based on PAT or PTT.

Accordingly, the present disclosure is directed to improved systems and methods of measuring blood pressure. In particular, the present disclosure is directed to a finger cuff assembly having an array of barometric pressure and one or more optical sensors with appropriate cuff actuation for measuring blood pressure of a patient. Thus, the finger cuff assembly of the present disclosure provides more convenient BP measurements than arm cuffs, without requiring a calibration step used in cuffless BP approaches. More specifically, the finger cuff assembly of the present disclosure includes an array of spatially localized barometric pressure sensors that are encapsulated in a soft elastomer to provide a force transfer medium between the area of the finger near a digital artery and the sensor array. In such embodiments, the array of barometric pressure sensors is used to account for differences in artery location and device positioning on the finger. Furthermore, in an embodiment, the array of miniature barometric pressure sensors also maps a pressure surface near the artery that provides several channels of data for accurate BP estimation, in contrast to a single cuff pressure channel from an air bladder wrapped around the finger. In addition, the finger cuff assembly of the present disclosure includes an optical sensor module having one or more optical sensors, such as a pulse oximeter, that is used to detect when a digital artery is occluded by the inflating air bladder.

Moreover, in an embodiment, the finger cuff assembly of the present disclosure contains a micropump to inflate an air bladder to increase pressure between the finger and the sensor array. As the pressure on the array of barometric pressure sensors increases, pulses from a digital artery are detected on several channels of the array. These pulses have distinct features, such as pulse amplitude and morphology, that vary throughout the pressure sweep; which are useful for BP estimation. Accordingly, the multi-channel and multi-modal (i.e., including both pressure and optical signals) design of the finger cuff assembly of the present disclosure is intended to increase accuracy of BP measurement, compared to previous measurement approaches at the finger.

With reference now to the Figures, example embodiments of the present disclosure will be discussed in further detail.

### Example Devices and Systems

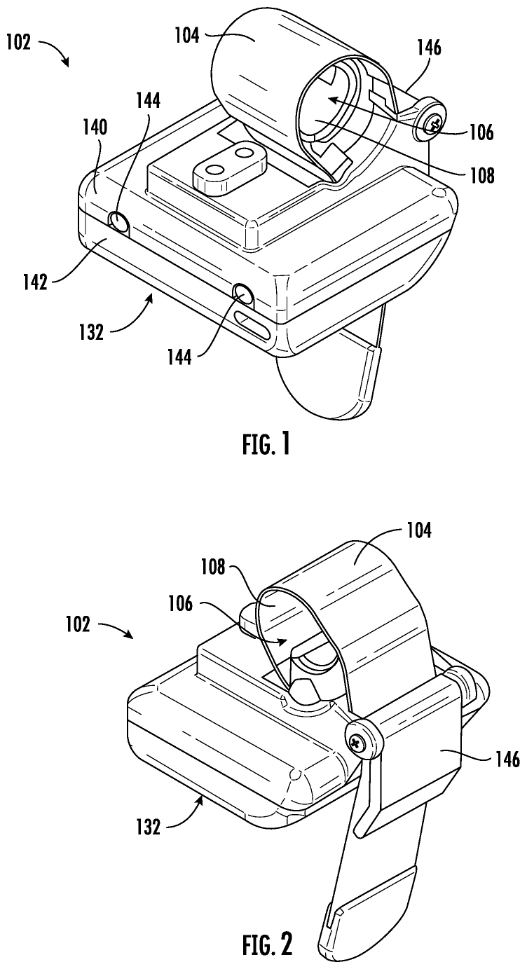

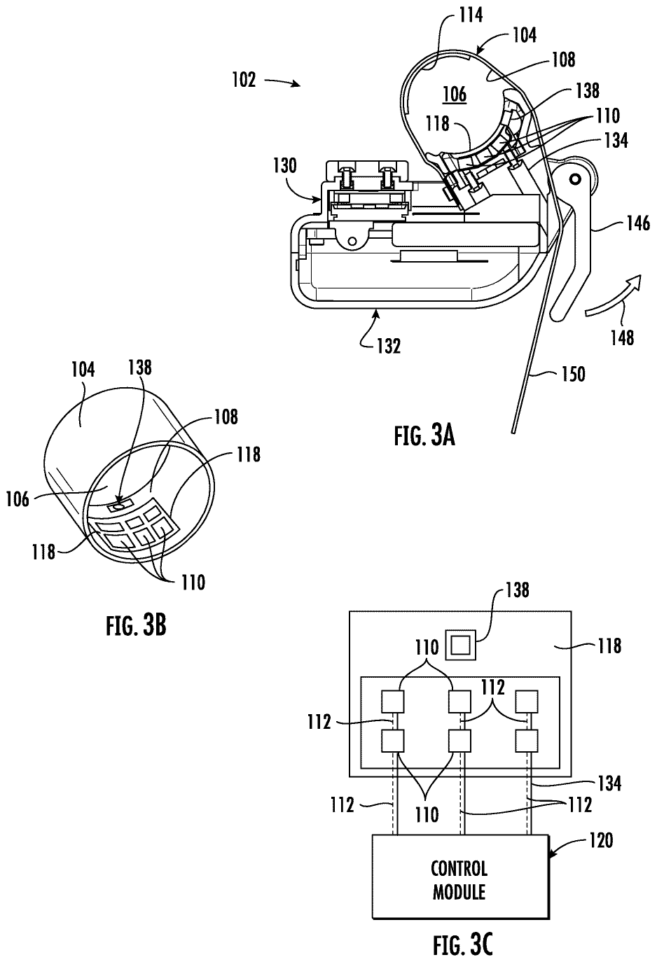

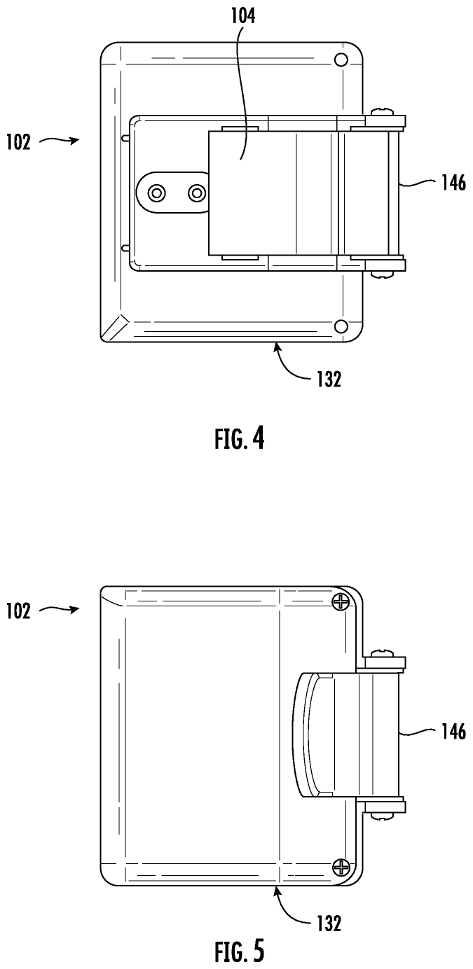

Referring now to the drawings, as shown in FIG. 1, a perspective view of an embodiment of a finger cuff assembly 102 for measuring blood pressure of a patient is illustrated according to the present disclosure. FIG. 2 illustrates another perspective view of the finger cuff assembly of FIG. 1, FIG. 3A illustrates a cross-sectional view of the finger cuff assembly of FIG. 1. FIG. 3B illustrates a front view of an array of sensors of the finger cuff assembly of FIG. 3A. FIG. 3C illustrates a perspective view of an array of sensors on an inner surface of the finger cuff assembly of FIG. 3A. FIG. 4 illustrates a top view of the finger cuff assembly of FIG. 1. FIG. 5 illustrates a bottom view of the finger cuff assembly of FIG. 1. In particular, as shown in FIGS. 1-4, the finger cuff assembly 102 includes a finger cuff 104 that may be attached to a patient's finger (e.g., by having the patient insert a finger into the finger cuff 104). Thus, the finger cuff 104 of the finger cuff assembly 102 is configured to measure a patient's blood pressure from a finger of the patient, which is further described herein.

More specifically, as shown in FIGS. 1-3C, the finger cuff 104 may also include a cavity 106 configured to receive the patient's finger, such that the finger cuff 104 extends around the finger. Furthermore, as shown in FIGS. 1-3C, the cavity 106 of the finger cuff 104 defines an inner surface 108 configured to engage or contact the finger of the patient. Further, as shown particularly in FIGS. 3A-3C, the finger cuff assembly 102 includes an array of barometric pressure sensors 110 arranged on the inner surface 108 of the cavity 106 of the finger cuff 104.

As used herein, an “array” of sensors generally refers to an arrangement of a plurality of sensors arranged on the inner surface 108 of the cavity 106 of the finger cuff 104 so as to cover more surface area of the finger. In particular embodiments, as shown in FIGS. 3B and 3C, the array of barometric pressure sensors 110 may be a grid of 3×2 sensors. In further embodiments, the array of barometric pressure sensors 110 may be a grid of 2×3 sensors, or 3×3 sensors, or 2×2 sensors, as well as more than two (2) sensors in any given row or column. In additional embodiments, the array of barometric pressure sensors 110 may be arranged across a portion of the inner surface 108 of the cavity 106 so as to cover a desired portion of a particular finger of the patient, such as about 40 degrees, more preferably about 50 degrees, more preferably about 60 degrees, still more preferably about 70 degrees, for improved data collection. In other words, by covering the desired portion of the finger of the patient, the sensor(s) 110 will be positioned appropriately with respect to one or more arteries in the finger regardless of the artery location that varies from person to person and/or regardless of rotational difference of the finger cuff 104 itself when placed on the finger.

Thus, the array of barometric pressure sensors 110, rather than a single sensor, is configured to account for differences in one or more artery locations in the finger, as well as device positioning variations on the finger. In particular, in any given finger of a particular patient or across patients, the finger is not symmetrical in nature and the arteries in the finger are not in the same location. Accordingly, in such embodiments, the array of barometric pressure sensors 110 are configured to map a pressure surface near an artery in the finger to provide a plurality of data channels 112 for measuring the blood pressure. More specifically, as shown in FIG. 3C, each sensor 110 in the array of barometric pressure sensors 110 corresponds to one of the data channels 112.

Furthermore, as shown in FIGS. 3A and 3B, the array of barometric pressure sensors 110 are encapsulated or covered in an elastomer material 118 to provide a force transfer medium between the finger and the array of barometric pressure sensors 110. In an embodiment, for example, the elastomer material 118 may be a silicone gel, silicone oil, mineral oil, or other suitable liquid encapsulated with a thermoplastic polyurethane (TPU) membrane containing the elastomer material 118 within the sensor module. It should be understood that “encapsulated” as used herein may generally refer to the array of barometric pressure sensors 110 being at least partially or fully enclosed in the elastomer material 118. In the illustrated embodiment, for example, the array of barometric pressure sensors 110 are covered in the elastomer material 118 such that the elastomer material 118, and not the sensors 110, directly contact the skin of the finger of the patient.

Referring particularly to FIGS. 3A-3C, the finger cuff assembly 102 may also include an optical sensor module having one or more optical sensors 138 arranged on the inner surface 108 of the cavity 106 of the finger cuff 104, e.g., adjacent to the array of barometric pressure sensors 110. In an embodiment, for example, the optical sensor(s) 138 directly contacts the skin of the finger of the patient and is not covered with the elastomer material 118. In such embodiments, for example, the optical sensor(s) 138 may be any of one or more pulse oximetry sensors (e.g., sensors used to measure the oxygen level of the blood) or one or more photoplethysmogram (PPG) sensors. Thus, in an embodiment, the optical sensor(s) 138 may be used to obtain a plethysmography signal from a digital artery of the finger. Accordingly, in such embodiments, the finger cuff assembly 102 of the present disclosure provides a multi-channel and multi-modal (i.e., having both pressure and optical signals) design that increases accuracy of the BP measurement compared to prior art measurement approaches at the finger. In particular, the combination of pressure and optical signals can be very useful for accurately estimating systolic blood pressure.

Moreover, as shown particularly in FIG. 3A, the finger cuff assembly 102 includes an inflatable bladder 114. More specifically, as shown in FIG. 3A, in an embodiment, the bladder 114 may be arranged opposite of the array of barometric pressure sensors 110 in the finger cuff 104. Furthermore, as shown in FIG. 3A, the inflatable bladder 114 may be inflated using, e.g., ambient air. Thus, in particular embodiments, for example, ambient air inflates the bladder 114 to increase a pressure on the array of barometric pressure sensors 110 against the finger of the patient. More specifically, in an embodiment, as shown in FIG. 3A, the finger cuff assembly 102 may include a micropump 130 for transferring the air from the environment to the inflatable bladder 114.

Moreover, as shown generally in FIGS. 1-5, the finger cuff assembly 102 also includes a housing 132 for housing various components of the assembly 102, such as a control module 120, the micropump 130, and/or any other component. In particular embodiments, as shown generally in FIGS. 1-4, the housing 132 may be secured to the finger cuff 104 to provide a compact design. Thus, in such embodiments, the housing 132 is configured to rest against the finger or a palm of the patient when the finger cuff 104 is positioned on the finger of the patient. In alternative embodiments, the housing 132 may be separate from the finger cuff 104. In such embodiments, for example, the housing 132 may be securable to a wrist or arm of the patient. Moreover, as shown, the housing 132 may be constructed of a unitary component or multiple components secured together at an interface. For example, in the illustrated embodiment of FIG. 1, the housing 132 is constructed of a first component 140 (or upper half) and a second component 142 (or lower half) secured together via one or more fasteners 144.

In additional embodiments, as shown in FIGS. 1-2, 3A, and 4-5, the finger cuff assembly 102 may further include a latching mechanism 146 secured to the housing for adjusting a dimension of the finger cuff 104. Thus, the dimension of the finger cuff 104 can be sized to fit any size finger and can be tightened or loosened accordingly. For example, in an embodiment, as shown in FIG. 3A, to adjust the finger cuff 104, which may be constructed of a strap-like material 150, the latching mechanism 146 can be rotated, e.g., in the direction indicated by arrow 148 such that the strap-like material 150 is loosened. When loosened, the patient can insert a finger and pull or push the strap-like material 150 to the appropriate dimension of the finger. Then, the latching mechanism 146 can be rotated back to its locked position (as shown in FIG. 3A), to secure the finger cuff 104 in place.

Furthermore, as shown in FIG. 3C, the finger cuff assembly 102 may include one or more cables 134 electrically coupling circuitry of the control module 120 to the array of barometric pressure sensors 110 and/or the optical sensor(s) 138.

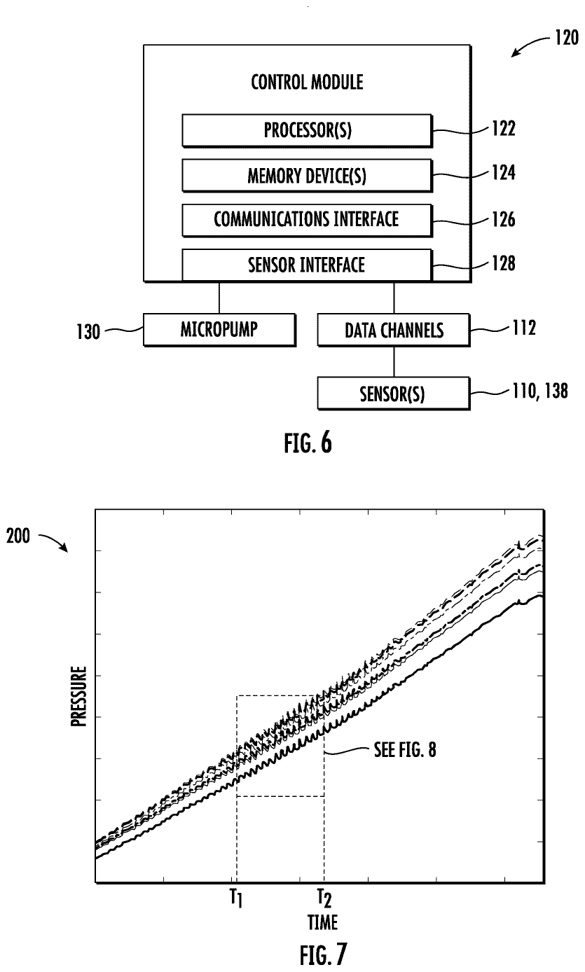

Referring now to FIG. 6, the finger cuff assembly 102 further includes a control module 120 having circuitry for receiving data from the plurality of data channels 112 and estimating the blood pressure using the data from the plurality of data channels 112. In another embodiment, the control module 120 is also configured to control a flow rate to the inflatable bladder 114, such as by controlling the micropump 130. Thus, in an embodiment, as the pressure on the array of barometric pressure sensors 110 increases, pulses from the artery are detected on the plurality of data channels 112 and used by the control module 120 for estimating the blood pressure. Moreover, in an embodiment, the circuitry of the control module 120 is further configured to store and/or process the data from the plurality of data channels 112 and estimate the blood pressure using the pulses from the artery detected on the plurality of data channels 112. In such embodiments, the pulses from the artery detected on the plurality of data channels 112 may have one or more distinct features that vary throughout a pressure sweep. For example, in an embodiment, the distinct feature(s) may include pulse amplitude and/or pulse morphology. Thus, the control module 120 can accurately estimate the blood pressure of the patient by observing these distinct feature(s) over time. Additionally, suitable digital signal processing (DSP) techniques may be used to filter out artifacts such as respiration and high frequency noise to allow for a more accurate blood pressure estimate.

In particular, as shown in FIG. 6, there is illustrated a block diagram of an embodiment of suitable components that may be included within the control module 120 according to the present disclosure. As shown, the control module 120 may include one or more processor(s) 122 and associated memory device(s) 124 configured to perform a variety of computer-implemented functions (e.g., performing the methods, steps, calculations and the like and storing relevant data as disclosed herein). Additionally, the control module 120 may also include a communications module 126 to facilitate communications between the control module 120 and the various components of the finger cuff assembly 102. Further, the communications module 126 may include a sensor interface 128 (e.g., one or more analog-to-digital converters) to permit signals transmitted from the sensors 110, 138 to be converted into signals that can be understood and processed by the processor(s) 122. It should be appreciated that the sensors 110, 138 may be communicatively coupled to the communications module 126 using any suitable means. For example, as shown in FIG. 6, the sensors 110, 138 are coupled to the sensor interface 128 via a wired connection. However, in other embodiments, the sensors 110, 138 may be coupled to the sensor interface 128 via a wireless connection, such as by using any suitable wireless communications protocol known in the art.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 124 may generally include memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 124 may generally be configured to store suitable computer-readable instructions that, when implemented by the processor(s) 122, configure the control module 120 to perform various functions to determine the blood pressure of a patient as described herein. For example, in an embodiment, the processor(s) 122 (and/or appropriate electronics, control circuitry, etc.) may be coupled to the array of barometric pressure sensors 110 and may utilize pressure changes measured by the array of barometric pressure sensors 110 as a reference for an arterial waveform of the patient that may be translated into the arterial blood pressure of the patient.

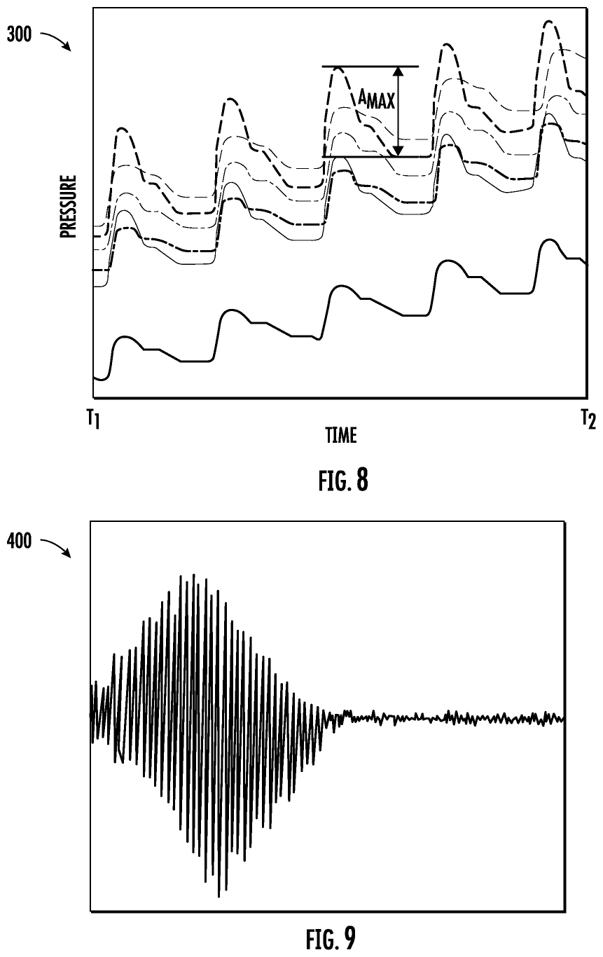

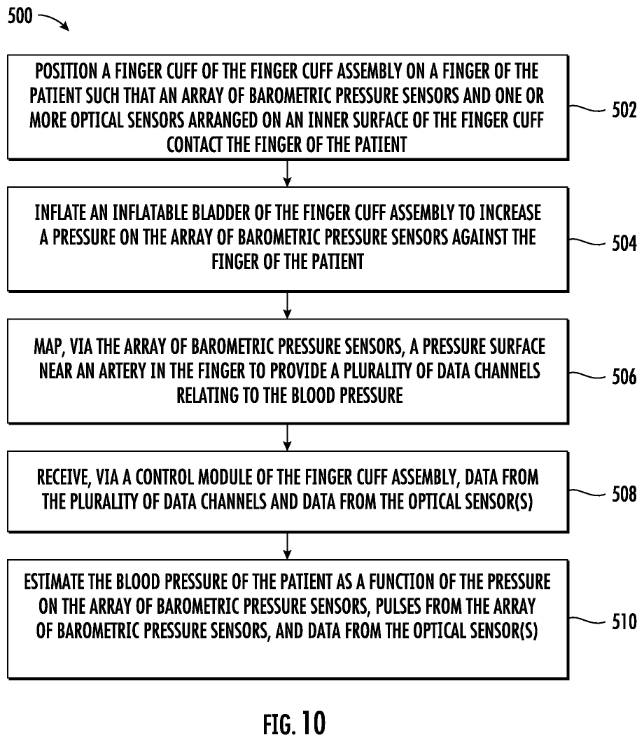

Referring now to FIGS. 7-9, various graphs are provided to illustrate sample data collected by systems and methods of the present disclosure, which can be used for estimating blood pressure of a patient via a finger cuff assembly. In particular, FIG. 7 illustrates a graph 200 of an embodiment of pressure (y-axis) versus time (x-axis) to illustrate a response of a sensor array of a finger cuff assembly as pressure is being applied to the sensor array according to the present disclosure. FIG. 8 illustrates a graph 300 of an embodiment of pressure (y-axis) versus time (x-axis) to illustrate a pulsatile response of a sensor array of a finger cuff assembly as pressure is being applied to the sensor array according to the present disclosure. In particular, FIG. 8 illustrates a close-up view of a portion of the graph 200 of FIG. 7. FIG. 9 illustrates a graph 400 of an embodiment of a response of an optical sensor, such as optical sensor 138 described herein, during a pressure sweep of a finger cuff assembly according to the present disclosure. In particular, as shown in FIGS. 7 and 8, between time T1 and T2, as pressure increases, data received from the data channels 112 oscillates up to a maximum pulse amplitude AMAX (FIG. 8), which generally corresponds to the mean blood pressure applied on an artery wall. Accordingly, this maximum pulse amplitude can be used as an estimate for an average blood pressure of the patient. Furthermore, the time instant when optical pulses disappear, as shown in FIG. 9, indicates that the digital artery is occluded, which generally corresponds to the systolic blood pressure condition.

Referring now to FIG. 10, a flow diagram of an embodiment of a method 500 for estimating blood pressure of a patient via a finger cuff assembly is illustrated. In general, the method 500 is described herein with reference to the finger cuff assembly 102 and various components thereof illustrated in FIGS. 1-6. However, it should be appreciated that the disclosed method may be implemented with any finger cuff assembly having any other suitable configurations. In addition, although FIG. 10 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown at (502), the method 500 includes positioning a finger cuff of the finger cuff assembly on a finger of the patient such that an array of barometric pressure sensors and one or more optical sensors arranged on an inner surface of the finger cuff contact the finger of the patient. As shown at (504), the method 500 includes inflating an inflatable bladder of the finger cuff assembly to increase a pressure on the array of barometric pressure sensors against the finger of the patient. As shown at (506), the method 500 includes mapping, via the array of barometric pressure sensors, a pressure surface near an artery in the finger to provide a plurality of data channels relating to the blood pressure. As shown at (508), the method 500 includes receiving, via a control module of the finger cuff assembly, data from the plurality of data channels and data from the optical sensor(s). As shown at (510), the method 500 includes estimating the blood pressure of the patient as a function of the pressure on the array of barometric pressure sensors, pulses from the artery detected on the plurality of data channels, and data from the optical sensor(s).

### Additional Disclosure

The various illustrative blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor 122 such the processor 122 can read information from, and write information to, the storage medium. In the alternative, the storage medium may be integral to the processor 122.

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A finger cuff assembly for measuring blood pressure of a patient, the finger cuff assembly comprising:
a finger cuff comprising a cavity defining an inner surface configured to engage a finger of the patient;
an array of barometric pressure sensors arranged on the inner surface of the cavity of the finger cuff, the array of barometric pressure sensors configured to map a pressure surface near an artery in the finger to provide a plurality of data channels for measuring the blood pressure;
one or more optical sensors arranged on the inner surface of the cavity of the finger cuff adjacent to the array of barometric pressure sensors;
an inflatable bladder arranged with the array of barometric pressure sensors, wherein inflating the inflatable bladder increases a pressure on the array of barometric pressure sensors against the finger of the patient; and
a control module comprising circuitry for receiving data from the plurality of data channels and estimating the blood pressure using the data from the plurality of data channels,
wherein, as the pressure on the array of barometric pressure sensors increases, pulses from the artery are detected on the plurality of data channels and used by the control module for estimating the blood pressure.

2. The finger cuff assembly of claim 1, wherein the array of barometric pressure sensors are encapsulated in an elastomer material to provide a force transfer medium between the finger and the array of barometric pressure sensors.

3. The finger cuff assembly of claim 2, wherein the elastomer material comprises a silicone gel, silicone oil, or mineral oil.

4. The finger cuff assembly of claim 1, wherein the one or more optical sensors comprises at least one of one or more pulse oximetry sensors, one or more thermal sensors, or one or more photoplethysmogram (PPG) sensors.

5. The finger cuff assembly of claim 1, further comprising a micropump for transferring ambient air to the inflatable bladder, wherein the circuitry of the control module is further configured to control the micropump.

6. The finger cuff assembly of claim 5, further comprising a housing for housing the control module and the micropump.

7. The finger cuff assembly of claim 6, wherein the housing is secured to the finger cuff and rests against at least one the finger or a palm of the patient when the finger cuff is positioned on the finger of the patient.

8. The finger cuff assembly of claim 6, wherein the housing is separate from the finger cuff.

9. The finger cuff assembly of claim 8, wherein the housing is securable to a wrist of the patient.

10. The finger cuff assembly of claim 6, further comprising one or more cables electrically coupling the circuitry of the control module to the array of barometric pressure sensors.

11. The finger cuff assembly of claim 1, wherein the circuitry of the control module is further configured to:
store the data from the plurality of data channels; and
estimate the blood pressure using the pulses from the artery detected on the plurality of data channels and stored in the circuitry of the control module.

12. The finger cuff assembly of claim 11, wherein the pulses from the artery detected on the plurality of data channels comprise one or more distinct features that vary throughout a pressure sweep.

13. The finger cuff assembly of claim 11, wherein the one or more distinct features comprise at least one of pulse amplitude or pulse morphology.

14. A method for estimating blood pressure of a patient via a finger cuff assembly, the method comprising:
positioning a finger cuff of the finger cuff assembly on a finger of the patient such that an array of barometric pressure sensors and one or more optical sensors arranged on an inner surface of the finger cuff contact the finger of the patient;
inflating an inflatable bladder of the finger cuff assembly to increase a pressure on the array of barometric pressure sensors against the finger of the patient;
mapping, via the array of barometric pressure sensors, a pressure surface near an artery in the finger to provide a plurality of data channels relating to the blood pressure;
receiving, via a control module of the finger cuff assembly, data from the plurality of data channels and data from the one or more optical sensors; and
estimating the blood pressure of the patient as a function of the pressure on the array of barometric pressure sensors and pulses from the artery detected on the plurality of data channels and the data from the one or more optical sensors.

15. The method of claim 14, wherein the array of barometric pressure sensors are encapsulated in an elastomer material to provide a force transfer medium between the finger and the array of barometric pressure sensors.

16. The method of claim 14, wherein inflating the inflatable bladder of the finger cuff assembly further comprises:
transferring, via a micropump, ambient air to the inflatable bladder to inflate the inflatable bladder; and
controlling, via the control module, the micropump to control a flow rate of the ambient air.

17. The method of claim 16, further comprising placing a housing of the finger cuff assembly against at least one the finger or a palm of the patient when the finger cuff is positioned on the finger of the patient.

18. The method of claim 17, further comprising:
securing one or more cables between the housing and the finger cuff for communicatively coupling the control module to the array of barometric pressure sensors.

19. The method of claim 14, wherein the one or more optical sensors comprises at least one of one or more pulse oximetry sensors, one or more thermal sensors, or one or more photoplethysmogram (PPG) sensors.

20. The method of claim 14, further comprising:
storing, via the control module, the data from the plurality of data channels; and
estimating the blood pressure using the stored data and the pulses from the artery detected on the plurality of data channels,
wherein the pulses from the artery detected on the plurality of data channels comprise one or more distinct features that vary throughout a pressure sweep, the one or more distinct features comprising at least one of pulse amplitude or pulse morphology.
